(19) United States
(12) Patent Application Publication
(10) Pub. No.: US 2021/0128864 A1
(43) Pub. Date: May 6, 2021

(54) OXYGEN RECOVERY DURING NASAL THERAPY

(71) Applicant: KONINKLIJKE PHILIPS N.V., EINDHOVEN (NL)

(72) Inventors: Francesco Vicario, Boston, MA (US); Nikolaos Karamolegkos, Boston, MA (US)

(21) Appl. No.: 17/021,369

(22) Filed: Sep. 15, 2020

Related U.S. Application Data
(60) Provisional application No. 62/931,331, filed on Nov. 6, 2019.

(51) Int. Cl. A61M 16/06; A61M 16/00; A61M 16/20

(52) U.S. Cl. CPC A61M 16/0672; A61M 16/0003; A61M 2016/0042; A61M 16/024; A61M 2016/0039; A61M 16/205

(57) ABSTRACT

A system includes an oxygen supply; one or more sensors configured to generate output signals conveying information as to whether the patient is in an inspiratory phase or in an expiratory phase; one or more valves; and a computer system. The one or more valves have a) a first configuration in which the one or more valves operate to recover an excess flow of the oxygen-enriched breathing gas during the inspiratory phase, and b) a second configuration in which the one or more valves vent an exhalation flow of the patient during the expiratory phase to atmosphere. One or more physical processors are programmed with computer program instructions which, when executed cause the computer system to provide input to the one or more valves based on the output signals, the provided input causing movement of the one or more valves between the first and second configuration.

### CROSS-REFERENCE TO RELATED APPLICATIONS

This patent application claims the priority benefit under 35 U.S.C. § 119(e) of U.S. Provisional Application No. 62/931,331, filed on Nov. 6, 2019, the contents of which are herein incorporated by reference.

### BACKGROUND

### 1. Field

The present patent application discloses a system and a method for providing oxygen therapy to a patient. Specifically, the system and the method of the present patent application is configured for oxygen recovery during high-flow nasal therapy to the patient.

### 2. Description of the Related Art

High-flow nasal therapy (HFNT) system has been gaining a lot of favor in hospital care settings. Increasingly often, it is used in place of or before escalation to non-invasive ventilation (NIV) system. It is also considered as an alternative to traditional (low-flow) long-term oxygen therapy (LTOT) system for nonhypercapnic hypoxemic respiratory failures. Compared to the LTOT system, the HFNT system offers a series of physiological and clinical benefits: 1) it enhances patient comfort due to the use of nasal prongs instead of masks; 2) it attains a more reliable delivery of oxygen (i.e., higher effective fraction of inspired oxygen, FiO₂) as a result of the high flow that exceeds patient demand and leads to a lesser entrainment of room air; 3) it provides more effective heat and humidification that facilitates removal of airway secretions, avoids airway desiccation, and enhances patient comfort; 4) it generates a positive end-expiratory pressure (PEEP) effect that enhances oxygenation, reduces intrinsic PEEP (if present), and reduces patient work of breathing; and 5) it washes out carbon dioxide from the nasopharyngeal deadspace, thereby leading to improved ventilation and oxygen delivery.

FIG. 1 depicts a HFNT system. It comprises the following components: i) air pump or blower for controlling the flow rate, ii) oxygen blender for controlling the FiO₂ level, iii) active humidifier that heats and humidifies the inspired air, and iv) nasal interface (prongs). Operation of the HFNT system is accomplished by selecting the desired flow rate (typical range: 5-60 liters per minute, lpm) and FiO₂ level (range: 21-100%) (in addition to the temperature setting). It is, hence, considered a device that is easy to operate, particularly in comparison to the NIV system.

Easy configuration and intuitive settings as well as enhanced patient comfort and mobility (e.g., the HFNT system is generally well tolerated even for prolonged periods of time) have been consistently underlined as strong indicators of the benefits in using the HFNT system for home care. Conditions, like mild to moderate chronic obstructive pulmonary disease (COPD) or obstructive sleep apnea (OSA), are a few cases that could benefit from the HFNT system.

In transitioning this technology from hospital to home care, one of the main technical challenges is related to oxygen consumption. Given the high rates of flow made available to the patient by the HFNT system (i.e., typically up to 60 lpm), the simultaneous administration of oxygen significantly reduces the duration of an oxygen cylinder/tank. For example, the HFNT system at 60 lpm with FiO₂ 50% consumes about 7 times more oxygen than the LTOT system with 100% oxygen at 3 lpm (i.e., with comparable oxygen uptake by the patient). Specifically, the HFNT system configured at 60 lpm with FiO₂ of 50% is delivering 30 lpm of oxygen, while a typical patient's oxygen uptake is around 0.5 lpm. Almost all the oxygen provided by the HFNT system is wasted.

In hospital settings, oxygen consumption is not an issue due to the continuous supply from the facility's oxygen filling plant. However, oxygen supply at home is not always secured. Limited availability of oxygen cylinders as well as high costs associated with replenishing them are major factors that have prevented the use of the HFNT system at home. Current alternatives, such as the LTOT system combined with an oxygen concentrator or high-flow systems without oxygen administration (room air delivery), share only a few of the benefits of the HFNT system. Even a combination of the HFNT system with an oxygen concentrator would not be able to sustain the high demand of oxygen. Another option for home care, which offers better oxygenation as compared to the LTOT system, is a reservoir cannula. This system has a membrane structure (the reservoir) and stores a small volume of oxygen-rich gas which is, in turn, available for the following inhalation as a short bolus of oxygen. However, such a system is only applicable to the LTOT system and there has not been any documented evidence that it improves the system's overall oxygen consumption. Clearly, a system that provides the HFNT system for use at home is an unmet need; the system's high requirements for oxygen cannot be easily met due to the limited oxygen supply at home.

Therefore, an improved system and method is provided to overcome the above-discussed problems and disadvantages.

### SUMMARY

Accordingly, one or more aspects of the present patent application relate to a system configured to provide oxygen therapy to a patient. The system comprises an oxygen supply configured to provide oxygen-enriched breathing gas to the patient during a breath cycle, the breath cycle comprising an inspiratory phase and an expiratory phase; a patient interface configured to deliver the oxygen-enriched breathing gas to a nasal cavity of the patient; one or more sensors configured to generate output signals conveying information as to whether the patient is in the inspiratory phase or in the expiratory phase; one or more valves operatively associated with the patient interface and the oxygen supply, the one or more valves having a) a first configuration in which the one or more valves operate to recover an excess flow of the oxygen-enriched breathing gas during the inspiratory phase, and b) a second configuration in which the one or more valves vent an exhalation flow of the patient during the expiratory phase to atmosphere; and a computer system that comprises one or more physical processors operatively connected with the one or more sensors and the one or more valves, the one or more physical processors being programmed with computer program instructions which, when executed cause the computer system to provide input to the one or more valves based on the output signals, the provided input causing movement of the one or more valves between the first configuration and the second configuration.

Another aspect of the present patent application relates to a method for providing oxygen therapy to a patient. The method is implemented by a computer system that comprises one or more physical processors executing machine readable instructions that, when executed, perform the method. The method comprises providing, using an oxygen supply and a patient interface, oxygen-enriched breathing gas to a nasal cavity of the patient during a breath cycle, the breath cycle comprising an inspiratory phase and an expiratory phase; obtaining, from one or more sensors, output signals conveying information related as to whether the patient is in the inspiratory phase or in the expiratory phase; and providing input to one or more valves based on the output signals, the provided input causing movement of the one or more valves between a first configuration and a second configuration. When in the first configuration, the one or more valves operate to recover an excess flow of the oxygen-enriched breathing gas during the inspiratory phase, and when in the second configuration, the one or more valves vent an exhalation flow of the patient during the expiratory phase to atmosphere.

Yet another aspect of the present patent application relates to a system for providing oxygen therapy to a patient. The system comprises an oxygen supply configured to deliver oxygen-enriched breathing gas to the patient during a breath cycle, the breath cycle comprising an inspiratory phase and an expiratory phase; a patient interface configured to deliver the oxygen-enriched breathing gas to a nasal cavity of the patient; and one or more valves operatively connected the patient interface and the oxygen supply, the one or more valves configured to move between a first configuration and a second configuration based on an increase in pressure generated during the expiratory phase. When the one or more valves are in the first configuration, the one or more valves operate to recover an excess flow of the oxygen-enriched breathing gas during the inspiratory phase, and when the one or more valves are in the second configuration, the one or more valves vent an exhalation flow of the patient during the expiratory phase to atmosphere.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 shows a prior art high-flow nasal therapy (HFNT) system;

FIG. 2 shows a system for providing oxygen therapy to the patient in accordance with an embodiment of the present patent application, wherein the system is a HFNT system;

FIG. 3 shows the HFNT system with oxygen recovery in accordance with an embodiment of the present patent application;

FIGS. 4 and 5 show exemplary sealed patient interfaces (i.e., nasal cannula) in accordance with an embodiment of the present patent application;

FIG. 6 shows the HFNT system with oxygen recovery and the sealed patient interface in accordance with an embodiment of the present patent application;

FIG. 7 shows an exemplary graphical representation of exemplary nasopharyngeal pressure waveforms during the HFNT under different flow rates in accordance with an embodiment of the present patent application;

FIG. 8 shows a method for providing the HFNT to the patient in accordance with an embodiment of the present patent application; and

FIG. 9 shows the HFNT system with oxygen recovery and the sealed patient interface in accordance with another embodiment of the present patent application.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

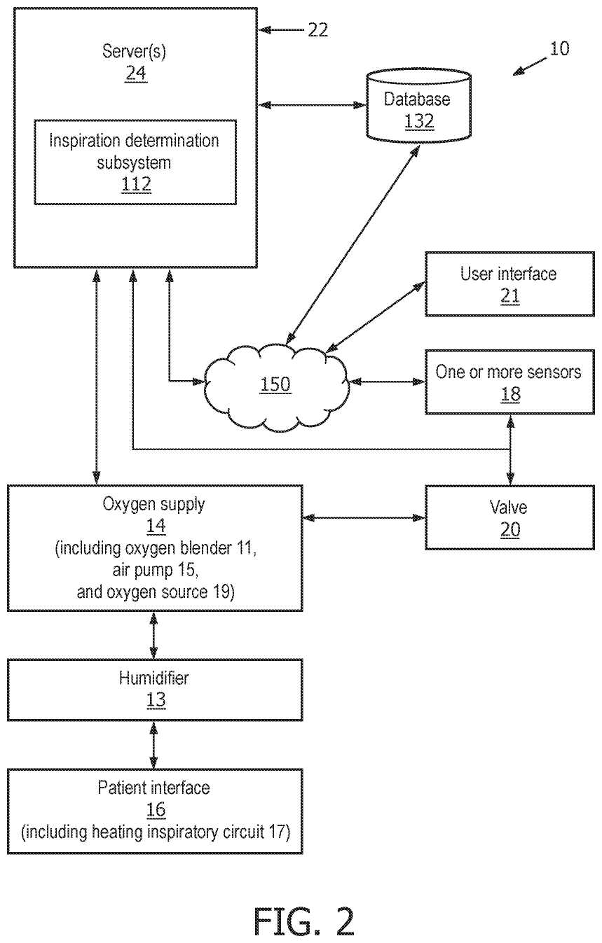

FIGS. 2, 3 and 6 schematically illustrate a system 10 configured to provide oxygen therapy to a patient 12. In some embodiments, system 10 is a high-flow nasal therapy (HFNT) system. System 10 comprises an oxygen supply 14 configured to provide oxygen-enriched breathing gas to patient 12 during a breath cycle; a patient interface 16 configured to deliver the oxygen-enriched breathing gas to a nasal cavity of patient 12; one or more sensors 18 configured to generate output signals conveying information as to whether patient 12 is in the inspiratory phase or patient 12 in the expiratory phase; one or more valves 20 operatively associated with patient interface 16 and oxygen supply 14; and a computer system 22 that comprises one or more physical processors 24 operatively connected with one or more sensors 18 and one or more valves 20. In some embodiments, the breath cycle comprises an inspiratory phase and an expiratory phase. In some embodiments, one or more valves 20 have a) a first configuration in which one or more valves 20 operate to recover an excess flow of the oxygen-enriched breathing gas during the inspiratory phase, and b) a second configuration in which one or more valves 20 vent an exhalation flow of patient 12 during the expiratory phase to atmosphere. In some embodiments, one or more physical processors 24 are programmed with computer program instructions which, when executed cause the computer system to provide input to one or more valves 20 based on the output signals. In some embodiments, the provided input causing movement of one or more valves 20 between the first configuration and the second configuration.

In some embodiments, when one or more valves 20 are in the first configuration, one or more valves 20 are configured to direct the excess flow of oxygen-enriched breathing gas during the inspiratory phase to oxygen supply 14. In some embodiments, when one or more valves 20 are in the first configuration, one or more valves 20 are configured to recirculate the excess flow of oxygen-enriched breathing gas during the inspiratory phase (i.e., instead of directing the excess flow of oxygen-enriched breathing gas during the inspiratory phase to oxygen supply 14) to patient 12. That is, in the short period of inhalation, the excess flow of oxygen-enriched breathing gas, during the inspiratory phase, does not have go all the way back to oxygen supply 14, but instead the excess flow of oxygen-enriched breathing gas, during the inspiratory phase, goes back to patient 12 during the next inhalation.

In some embodiments, as shown in FIG. 9, one or more valves 20 of system 10 include a second valve 119. In some embodiments, second valve 119 is configured, during inhalation, to direct the gas (i.e., Q_{s, inh}) to patient 12. In some embodiments, second valve 119 is configured, during exhalation, to direct the gas (i.e., Q_{s, exh}) back to system 10/blender 11 (via oxygen sensor 23) rather than to patient 12. In some embodiments, system 10 is configured to not deliver air/gas to patient 12 during exhalation. For example, such a configuration may be used an oxygen-conservation option a) during extreme cases, like close to the end of life of the oxygen supply/cylinder) or b) when some benefits (e.g., CO₂ clearance, small PEEP, etc. provided to patient by continuous flow of air during HFNT) are not necessary to a specific class of patients and during portion of the day, like at night. In some embodiments, instead of one second valve 119, one or more valves may be used to achieve this configuration/function (i.e., not deliver air/gas to patient 12 during exhalation).

In some embodiments, second valve 119 is configured to prevent mixing of the exhaled gas and the excess gas during the exhalation phase. In some embodiments, system 10 is configured to detect exhalation phase and to control valve 119 to direct the gas (i.e., Q_{s, exh}) back to system 10/blender 11 (via oxygen sensor 23) rather than to patient 12. In some embodiments, by having second valve 119 to perform such an operation is equivalent to stopping the gas flow and/or stopping the blower. For example, in some embodiments, blower always on and valve 119 is used just re-direct the flow back to system 10/blender 11 (via oxygen sensor 23) rather than to patient 12 during exhalation.

In some embodiments, referring to FIG. 9, during the inhalation phase, valve 119 is configured to only direct air/gas (i.e., Q_{s, inh}) to patient 12. That is, during the inhalation phase, valve 119 is configured to prevent air/gas to flow towards oxygen sensor 23 and/or valve 20 (i.e., that flow path is closed).

In some embodiments, during the exhalation phase, valve 119 is configured to only direct air/gas (i.e., Q_{s, exh}) to oxygen sensor 23 (and then to oxygen blender 11). That is, in some embodiments, during the exhalation phase, air/gas (i.e., Q_{s, exh}) cannot pass through valve 20 (i.e., that flow path is closed) and can only be directed to oxygen sensor 23 (and then to oxygen blender 11). Also, during the exhalation phase, valve 119 is configured to prevent air/gas to flow to patient 12 (i.e., that flow path is closed).

In some embodiments, referring to FIG. 9, during the inhalation phase, valve 20 is configured to only direct excess air/gas (i.e., Q_{e, inh}) to oxygen sensor 23 (and then to oxygen blender 11). That is, during the inhalation phase, valve 20 is configured to prevent air/gas (i.e., Q_{e, exh}) towards ambient (i.e., that flow path is closed). In some embodiments, during the exhalation phase, valve 20 is configured to only direct air/gas (i.e., Q_{e, exh}) to ambient. That is, during the exhalation phase, valve 20 is configured to prevent air/gas towards oxygen sensor 23 (i.e., that flow path is closed).

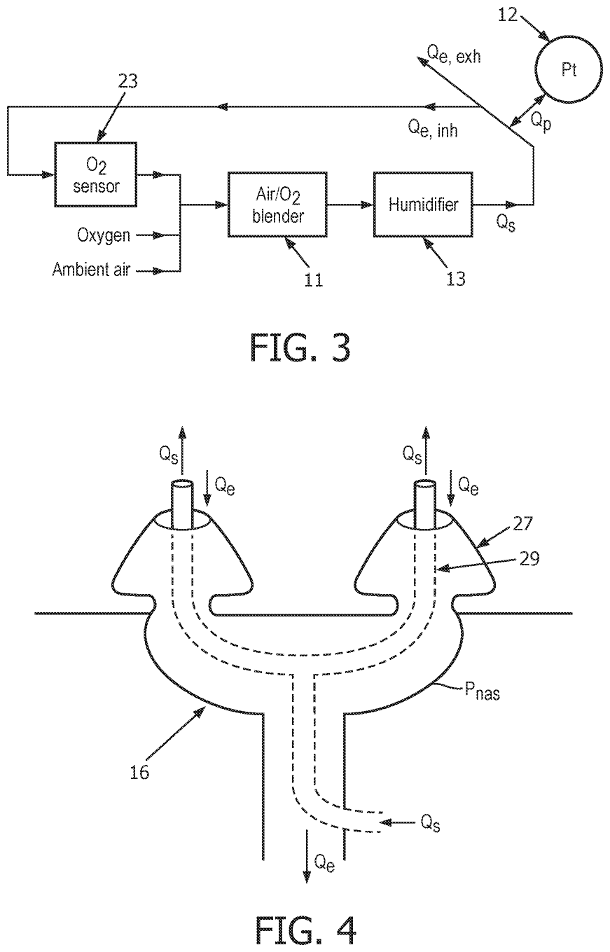

In some embodiments, in this patent application, the patient may be interchangeably referred to as a consumer, a user, an individual or a subject. In some embodiments, in this patent application, patient 12 may also be interchangeably referred to as patient Pt (see e.g., FIG. 3). In some embodiments, in this patent application, high-flow nasal therapy (HFNT) may be interchangeably referred to oxygen therapy. In some embodiments, in this patent application, the inhalation may be interchangeably referred to as inspiratory phase. In some embodiments, in this patent application, the exhalation may be interchangeably referred to as expiratory phase. In some embodiments, hardware processors may be interchangeably referred to as physical processors. In some embodiments, machine readable instructions may be interchangeably referred to as computer program instructions.

The present patent application provides systems and methods for oxygen recovery during the high-flow nasal therapy. High-flow nasal therapy (HFNT) is generally configured to supply high gas flow rates that exceed patient demands and to comfortably facilitate the patient's breathing activity. When combined with an oxygen therapy, oxygen consumption is very high. This may be a deterrent for use by patients in home care. The present patent application describes a novel HFNT system that enables the recovery of the oxygen-rich breathable gas that is otherwise dispersed to ambient by the current/prior art HFNT systems. The proposed system promises lower oxygen consumption, thereby allowing improved efficiency and reduced costs for home users. In some embodiments, recovery of the oxygen-rich excess flow during inhalation, Q_{e,inh} (see e.g., FIG. 3), requires two technical features: 1) a sealed nasal interface that enables delivery and recovery of gas, thereby eliminating gas dispersion to ambient, and 2) an element (passive or active) that allows the recovery of gas only during inhalation and prevents the expired carbon dioxide to return to patient 12. In some embodiments, as described in detail in the discussions below, the element that allows the recovery of oxygen-enriched breathing gas only during the inhalation phase and prevents the expired carbon dioxide to return to patient 12 includes a valve element.

The system and method of the present patent application focuses on an improved and more oxygen-efficient HFNT system that reduces oxygen consumption without requiring changes in the system's settings (e.g., reduction in flow rate or FiO₂). This is accomplished by recovering the gas/inhaled oxygen/inspired oxygen that is otherwise dispersed to the ambient by a traditional/current/prior art HFNT system. Gas dispersion to ambient is common in a traditional/prior art HFNT system. One of the reasons why in the HFNT system the set/predetermined flow rate is higher than the patient's demand is to avoid room air entrainment. Thus, ensuring more reliable delivery of the desired/predetermined FiO₂. Fraction of inspired oxygen (FiO₂) is the molar or volumetric fraction of oxygen in the inhaled gas. As a result, the majority of the flow, which is delivered to patient interface 16, is rich in oxygen, and is directly going to ambient without being inhaled by patient 12. Also, the gas exhaled by the patient is high in oxygen fraction as well, although contaminated with carbon dioxide from the patient's metabolic activity. The proposed system is based on the fact that the HFNT system is configured to provide flow above the patient's demand. This excess flow, which is rich in oxygen, can be exploited instead of wasted and dispersed to the ambient, as typically done in the current/prior art HFNT systems.

FIG. 2 shows a high-level schematic of the HFNT system with oxygen recovery in accordance with an embodiment of the present patent application.

In some embodiments, valve 20 is an electronic valve or a solenoid valve. In some embodiments, valve 20 is configured to operate via a computer system. In some embodiments, as explained in detail below, valve 20 is a passive valve and does not operate via a computer system. In some embodiments, valve 20 is in fluid communication with patient interface 16 and oxygen supply 14. In some embodiments, valve 20 is configured to be controlled by output signals/commands received from computer system 22. In some embodiments, valve 20 is configured to be controlled by output signals/commands received from computer system 22 to be in the first configuration in which valve 20 directs an excess flow of the oxygen-enriched breathing gas during the inspiratory phase to oxygen supply 14. In some embodiments, valve 20 is configured to be controlled by output signals/commands received from computer system 22 to be in the second configuration in which valve 20 vents an exhalation flow of patient 12 during the expiratory phase to atmosphere.

In some embodiments, patient interface 16 is operatively coupled to the patient/delivery circuit to communicate the oxygen-enriched breathing gas to the nasal cavity/airway of patient 12. In some embodiments, patient interface 16 is also configured to recover the excess gases during both the inhalation phase and the exhalation phase. In some embodiments, one or more valves 20, 119 and/or patient interface 16 are configured to recover the excess gases during both the inhalation phase and the exhalation phase. That is, during the inhalation phase, the excess gas Q_{e, inh} (as shown in FIGS. 3, 6 and 9) is recovered by one or more valves 20 and/or sealed patient interface 16. During the exhalation phase, gas Q_{s, exh} (as shown in FIG. 9) is recovered by one or more valves 20 (e.g., valve 119) and/or sealed patient interface 16. The recovered gas, during both the inhalation phase and the exhalation phase, is directed to oxygen supply 14/blender 11 via oxygen sensor 23.

In some embodiments, delivery circuit may include a conduit and/or patient interface 16. Delivery circuit may sometimes be referred to as patient interface 16. In some embodiments, the conduit may include a flexible length of hose, or other conduit, either in single-limb or dual-limb configuration that places patient interface 16 in fluid communication with oxygen supply 14. In some embodiments, the conduit forms a flow/fluid path through which the flow of oxygen-enriched breathing gas is communicated between patient interface 16 and oxygen supply 14.

In some embodiments, oxygen supply 14 includes an oxygen blender 11, an air pump 15, an oxygen source 19, and a humidifier 13. In some embodiments, oxygen supply 14 includes an oxygen blender 11, an air pump 15, and an oxygen source 19. Each of oxygen blender 11, air pump 15, oxygen source 19, and humidifier 13 are described in detail in the discussions below.

In some embodiments, the conduit of the delivery circuit forms a flow/fluid path through which the flow of oxygen-enriched breathing gas is communicated between air pump 15 and humidifier 13.

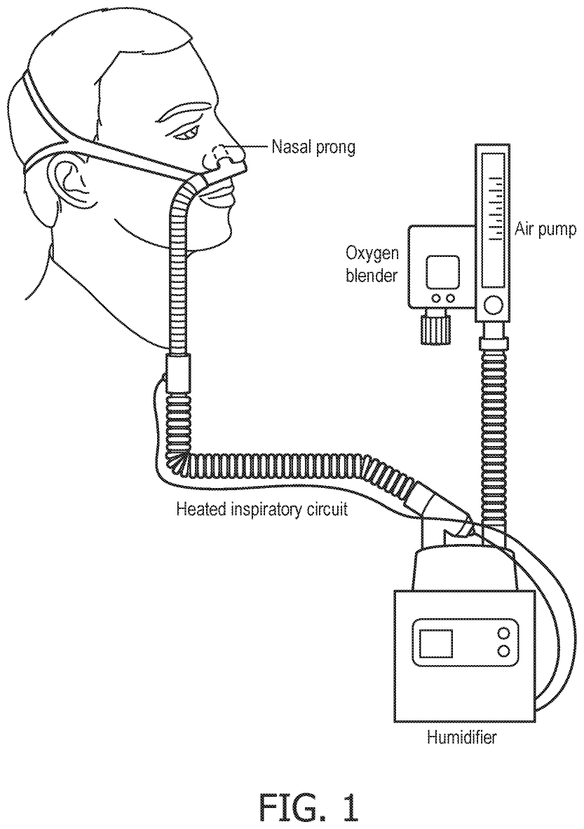

In some embodiments, system 10 includes heated respiratory circuit 17 that is configured to form a flow/fluid path through which the flow of oxygen-enriched breathing gas is communicated between patient interface 16 and humidifier 13. In some embodiments, heated respiratory circuit 17 is part of patient interface 16. In some embodiments, heated respiratory circuit 17 is part of delivery circuit. In some embodiments, heated respiratory circuit 17 is similar to that shown in FIG. 1.

In some embodiments, patient interface 16 may be configured to deliver oxygen-enriched breathing gas to the nasal cavity/airway of patient 12. As such, patient interface 16 may include any appliance/device suitable for this function. In some embodiments, patient interface 16 is configured to be removably coupled with another interface being used to deliver oxygen therapy to patient 12. For example, patient interface 16 may be configured to engage with and/or be inserted into other interface appliances/devices. In some embodiments, patient interface 16 may be configured to engage the airway/the nasal cavity of patient 12 without an intervening device. In some embodiments, patient interface 16 may include one or more of an a nasal cannula, nasal interface, nasal prongs, nasal pillows, a nasal mask, a nasal/oral mask, a full-face mask, a total facemask, and/or other interface devices that communicate a flow of oxygen-enriched breathing gas with an airway/a nasal cavity of patient 12. The present patent application is not limited to these examples, and contemplates delivery of the oxygen-enriched breathing gas to patient 16 using any subject interface.

In some embodiments, one or more sensors 18 are configured to generate output signals conveying information as to whether patient 12 is in the inspiratory phase or patient 12 is in the expiratory phase.

In some embodiments, one or more sensors 18 are configured to generate output signals conveying information related to one or more parameters of the flow of oxygen-enriched breathing gas and the respiration of patient 12. As another example, the information may be obtained from one or more monitoring devices (e.g., flow monitoring device, pressure monitoring device, or other monitoring devices). In some embodiments, one or more monitoring devices and associated sensors 18 may be configured to monitor flow of oxygen-enriched breathing gas being delivered to the nasal cavity of patient 12. In some embodiments, one or more monitoring devices and associated sensors 18 may be configured to monitor pressure of oxygen-enriched breathing gas being delivered to the nasal cavity of patient 12. These monitoring devices may include one or more sensors 18, such as pressure sensors, pressure transducers, flow rate sensors, or other sensors. Sensors 18 may, for instance, be configured to obtain information of the patient (e.g., airway pressure, nasopharyngeal pressure, airway flow, or any other airway parameters) or other information related to the patient.

In some embodiments, each of one or more sensors 18 include a transmitter for sending signals and a receiver for receiving the signals. In some embodiments, one or more sensors 18 are configured to communicate wirelessly with computer system 22. As shown in FIG. 2, in some embodiments, sensor 18 is configured to be operatively connected with computer system 22 and/or one or more physical processors 24 of computer system 22. In some embodiments, one or more sensors 18 are configured to communicate with oxygen supply 14, patient interface 16, and valve 20. In some embodiments, one or more sensors 18 are in communication with a database 132. In some embodiments, the information related to one or more parameters of the flow of oxygen-enriched breathing gas and the respiration of subject 12 may be obtained from the database 132 that is being updated in real-time by one or more sensors 18. In some embodiments, one or more sensors 18 are in fluid communication with breathing or patient passage/circuit/tubing/conduit of system 10.

In one scenario, a monitoring device may obtain information (e.g., based on information from one or more sensors 18), and provide information to computer system 22 (e.g., comprising server 24) over a network (e.g., network 150) for processing. In another scenario, upon obtaining the information, the monitoring device may process the obtained information, and provide processed information to computer system 22 over a network (e.g., network 150). In yet another scenario, the monitoring device may automatically provide information (e.g., obtained or processed) to computer system 22 (e.g., comprising server 24). In some embodiments, sensors 18 may be placed close to the nostrils (or other nasal interfaces) of the patient and/or at the system's outlet or other locations, with appropriate compensation algorithms to estimate the corresponding airflow and pressure in proximity of the patient's nostrils (or other nasal interfaces). In some embodiments, server 24 includes one or more physical/hardware processors 24. In FIG. 2, database 132 is shown as a separate entity, but, in some embodiments, database 132 could be part of computer system 22.

In some embodiments, system 10 includes one or more hardware processors 24 operatively connected with oxygen supply 14, patient interface 16, valve 20 and one or more sensors 18. As shown in FIG. 2, system 10 may comprise server 24 (or multiple servers 24). In some embodiments, server 24 comprises inspiration determination subsystem 112, user input subsystem 114, control subsystem 116 or other components or subsystems.

As will be clear from the discussions above and below, in some embodiments, system 10 includes computer system 22 that has one or more physical/hardware processors 24 programmed with computer program/machine readable instructions that, when executed cause computer system 22 to obtain information or data from one or more sensors 18.

In some embodiments, inspiration determination subsystem 112 is configured to determine as to whether patient 12 is in the inspiratory phase or patient 12 is in the expiratory phase based on the information in the output signals generated by one or more sensors 18. In some embodiments, inspiration determination subsystem 112 includes inspiration sensing algorithms 25. In some embodiments, inspiration determination subsystem 112 is configured to determine the one or more parameters of the flow of oxygen-enriched breathing gas and the respiration of subject 12 based on the information in the output signals generated by one or more sensors 18. In some embodiments, the one or more parameters of the flow of oxygen-enriched breathing gas and the respiration of patient 12 may include nasopharyngeal pressure, pressure at the nasal interface, or other respiratory airway parameters.

In some embodiments, one or more sensors 18 includes a pressure manometer or other pressure sensor that is incorporated with the nasal interface for the measurement of the nasopharyngeal pressure (Pₙₐₛ). In some embodiments, pressure readings at the nasal interface are used for identifying inspiratory and expiratory phases of a breath cycle of patient 12.

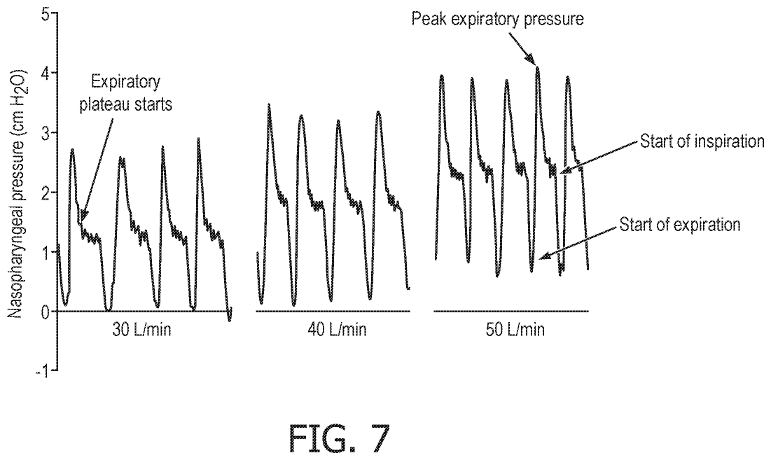

For instance, FIG. 7 shows exemplary nasopharyngeal pressure waveforms during the HFNT under different flow rates (i.e., 30 lpm or L/min; 40 L/min; and 50 L/min). In some embodiments, the graph shown in FIG. 7 has pressure (measured in cm H2O) on the Y-axis. In some embodiments, the graph shown in FIG. 7 has flow rate (measured in liters per min (L/min)) on the X-axis. The graph shown in FIG. 7 is intended to be an example. Referring to FIG. 7, start of the inspiration phase is generally indicated by the sudden decrease in pressure from the plateau level attained during the end of the previous expiratory phase. In turn, the expiration phase is marked by the reversal of the direction of change (from negative to positive) as the pressure reaches its minimal (intra-breath) value. In some embodiments, one or more hardware processors 24 are configured such that the pressure values are determined for individual breaths in an ongoing manner during the oxygen therapy.

In some embodiments, user input subsystem 114 is configured to receive user/patient's input. In some embodiments, the user input may include desired flow rate; desired FiO₂ level; desired temperature setting, etc. In some embodiments, one or more hardware processors 24 are configured to operate system 10 based on the desired user input.

Referring to FIG. 2, system 12 generally also comprises air/oxygen blender 11, humidifier 13, air pump 15, oxygen source 19 and heated respiratory circuit 17.

In some embodiments, oxygen source 19 is configured to supply the oxygen to oxygen blender 11. In some embodiments, oxygen source 19 is an oxygen tank or an oxygen cylinder, which stores compressed, oxygen enriched gas.

In some embodiments, air pump 15 is configured to control flow rate of the oxygen-enriched breathing gas being delivered to the nasal cavity of patient 12. In some embodiments, air pump 15 is a variable speed pump or a variable speed blower. In some embodiments, air pump 15 may include valves, stepper motor, flow rate sensors, and drive electronics. In some embodiments, air pump 15 is configured to control flow rate of the oxygen-enriched breathing gas being delivered to the nasal cavity of patient 12 as determined by output signals/commands received from computer system 22. In some embodiments, air pump 15 is configured to control flow rate of the oxygen-enriched breathing gas being delivered to the nasal cavity of patient 12 based on the patient's input. In some embodiments, air pump 15 is similar to that shown in FIG. 1.

In some embodiments, oxygen blender 11 is interchangeably referred to as an oxygen/air blender. In some embodiments, oxygen blender 11 is configured to control FiO₂ level of the oxygen-enriched breathing gas being delivered to the nasal cavity of patient 12. In some embodiments, oxygen blender 11 is configured to mix/blend oxygen from oxygen source 19 and ambient air to the desired concentration as determined by output signals/commands received from computer system 22. In some embodiments, oxygen blender 11 is configured to mix/blend oxygen from oxygen source 19 and ambient air to the desired oxygen fraction based on the patient's or physician's/caregiver's input according to a prescribed treatment plan. In some embodiments, oxygen blender 11 may include valves, stepper motor, and drive electronics. In some embodiments, oxygen blender 11 is similar to that shown in FIG. 1.

In some embodiments, oxygen blender 11 is configured to receive the recovered excess flow of the oxygen-enriched breathing gas during the inspiratory phase. In some embodiments, oxygen blender 11 is configured to receive the recovered excess flow of the oxygen-enriched breathing gas during the inspiratory phase via an inlet. In some embodiments, oxygen blender 11 is configured to receive the recovered excess flow of the oxygen-enriched breathing gas, during the inspiratory phase, after the excess flow of the oxygen-enriched breathing gas passed oxygen sensor 23.

In some embodiments, referring to FIG. 6, delivery circuit is disposed between valve 20 and oxygen/air blender 11. In some embodiments, a conduit of the delivery circuit forms a flow/fluid path through which the excess flow of oxygen-enriched breathing gas is communicated between valve 20 and oxygen/air blender 11. In some embodiments, oxygen sensor 23 is disposed in the delivery circuit disposed between valve 20 and oxygen/air blender 11.

In some embodiments, oxygen blender 11 is configured to mix/blend oxygen from oxygen source 19 and/or the recovered excess flow of the oxygen-enriched breathing gas during the inspiratory phase with the ambient air to the desired oxygen fraction as determined by output signals/commands received from computer system 22. In some embodiments, oxygen blender 11 is configured to mix/blend oxygen from oxygen source 19 and/or the recovered excess flow of the oxygen-enriched breathing gas during the inspiratory phase with the ambient air to the desired oxygen fraction based on the patient's or physician's input according to a prescribed treatment plan.

In some embodiments, humidifier 13 is an active humidifier. In some embodiments, humidifier 13 is configured to heat and humidify the oxygen-enriched breathing gas being delivered to the nasal cavity of patient 12. In some embodiments, humidifier 13 is configured to heat and humidify the oxygen-enriched breathing gas being delivered to the nasal cavity of patient 12 as determined by output signals/commands received from computer system 22. In some embodiments, humidifier 13 is configured to heat and humidify the oxygen-enriched breathing gas being delivered to the nasal cavity of patient 12 based on the patient's input. In some embodiments, humidifier 13 may include liquid chamber (that holds liquid or receives liquid from a liquid source), heater/heat source (that heats the liquid into a vapor that humidifies gas the oxygen-enriched breathing gas being delivered to the nasal cavity of patient 12), inlet/outlet, valves, stepper motor, and drive electronics. In some embodiments, humidifier 13 is similar to that shown in FIG. 1.

In some embodiments, system 10 comprises a user interface 21 configured to enable patient 12 to select, according to a prescribed treatment plan, a predetermined flow rate of the oxygen-enriched breathing gas being delivered to the nasal cavity of patient 12; a predetermined FiO₂ level of the oxygen-enriched breathing gas being delivered to the nasal cavity of patient 12; and a predetermined temperature of the oxygen-enriched breathing gas being delivered to the nasal cavity of the patient.

In some embodiments, the predetermined flow rate of the oxygen-enriched breathing gas being delivered to the nasal cavity of patient 12 using system 10 is in the range between 5 and 60 liters per minute (lpm). In some embodiments, the predetermined FiO₂ level of the oxygen-enriched breathing gas being delivered to the nasal cavity of patient 12 using system 10 is in the range between 21 and 100%. In some embodiments, the predetermined temperature of the oxygen-enriched breathing gas being delivered to the nasal cavity of patient 12 using system 10 is in the range between 31 and 37 Celsius.

In some embodiments, user interface 21 may be configured to provide an interface between system 10 and patient 12 through which patient 12 can provide information to and receive information from system 10. This enables data, results, and/or instructions and any other communicable items, collectively referred to as “information,” to be communicated between patient 12 and system 10. Examples of interface devices suitable for inclusion in user interface 21 include a keypad, buttons, switches, a keyboard, knobs, levers, a display screen, a touch screen, speakers, a microphone, an indicator light, an audible alarm, and a printer. Information may be provided to patient 12 by user interface 21 in the form of auditory signals, visual signals, tactile signals, and/or other sensory signals. It is to be understood that other communication techniques, either hard-wired or wireless, are also contemplated herein as the user interface. For example, in one embodiment, user interface 21 may be integrated with a removable storage interface provided by electronic storage 132. In this example, information is loaded into system 10 from removable storage (e.g., a smart card, a flash drive, a removable disk, etc.) that enables the user(s) to customize system 10. Other exemplary input devices and techniques adapted for use with system 10 as user interface include, but are not limited to, an RS-232 port, RF link, an IR link, modem (telephone, cable, Ethernet, internet or other). In short, any technique for communicating information with system 10 is contemplated as the user interface.

In some embodiments, referring to FIGS. 3-6, patient flow is represented as Qₚ, total system flow is represented as Qₛ; excess flow during inhalation/inspiratory phase is Q_{e,inh}; and system flow during exhalation/expiratory phase is Q_{e,exh}.

In some embodiments, Qₛ is the oxygen-enriched flow/breathing gas that is delivered to patient interface 16 at a constant rate throughout the breath cycle. In some embodiments, the breath cycle includes the inspiratory phase and the expiratory phase. In some embodiments, the oxygen from oxygen source 19 blends with the ambient air in air/oxygen blender 11. This blend of oxygen-enriched flow/breathing gas then passes through humidifier 13 so that the oxygen-enriched breathing gas being delivered to the nasal cavity of patient 12 is heated and humidified.

In some embodiments, Qₚ is the flow inspired (i.e., referred to as patient demand, Qₛ≥Qₚ) or expired by patient 12.

In some embodiments, Q_{e,inh} is the excess flow during inhalation and Q_{e,exh} is the excess/total flow during exhalation. In other words, Q_{e,inh}=Qₛ−Qₚ during inhalation/inspiratory phase (assuming zero room air entrainment) and Q_{e,exh}=Qₚ+Qₛ during exhalation/expiratory phase.

In some embodiments, Q_{e,inh} does not contain any carbon dioxide and its oxygen fraction is determined by the desired FiO₂ setting of the HFNT system. On the other hand, Q_{e,exh} is a mixture of the patient's exhalation flow and Qₛ. Hence, it contains some fraction of carbon dioxide.

Referring to FIG. 3, system 10 comprises an oxygen sensor 23. In some embodiments, oxygen sensor 23 is configured to measure oxygen fraction in the excess flow of the oxygen-enriched breathing gas, during the inspiratory phase, directed to oxygen supply 14. In some embodiments, oxygen sensor 23 is configured to be connected to the recovery line (i.e., that recovers the oxygen-enriched excess flow during inhalation) to measure the oxygen fraction in the excess flow during inhalation, Q_{e,inh}.

In some embodiments, system 10 also includes a dryer to maintain the correct dew point and to prevent condensation. In some embodiments, system 10 also includes a carbon dioxide scrubber to remove any excess carbon dioxide.

In some embodiments, the recovery line is then fed into air/oxygen blender 11 that mixes the recovered gas, ambient air, and pure oxygen from oxygen source (e.g., cylinder) 19 in order to deliver gas according to the system's FiO₂ setting.

In some embodiments, given that typically the inhalation time is about 30% of the duration of a breath cycle, the proposed system reduces the overall consumption of oxygen by 30% compared to the existing HFNT systems. In some embodiments, the duration of oxygen tank/source 19 would increase by the same percentage and the costs associated with it (or with an oxygen concentrator) would similarly reduce by 30%.

In some embodiments, patient interface 16 includes a seal structure (e.g., nasal pillows 27) constructed and arranged to form a seal with a region surrounding an entrance to the patient's nasal cavity such that the oxygen-enriched breathing gas is delivered to the nasal cavity of patient 12 and the excess flow of the oxygen-enriched breathing gas, during the inspiratory phase, is directed to oxygen supply 14. In some embodiments, patient interface 16 includes a sealed nasal interface. As illustrated in FIGS. 4-6, HFNT system 10 with oxygen recovery includes sealed nasal interface 16 for allowing delivery as well as recovery of excess oxygen-enriched breathing gas. Examples of such an interface are depicted in FIGS. 4-6.

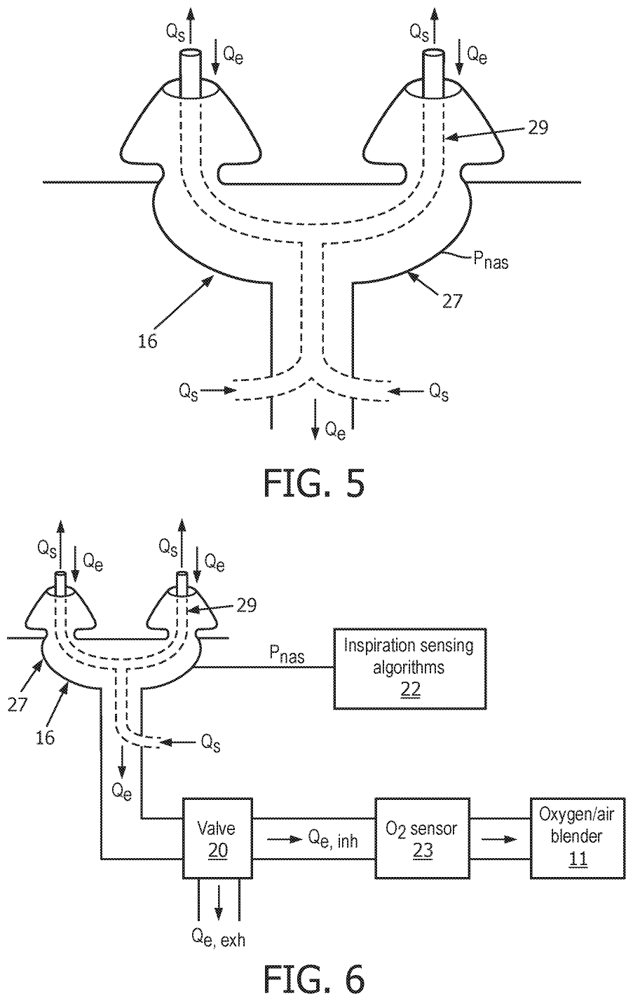

FIG. 4 shows sealed nasal interface 16 with a single gas supply tube, whereas FIG. 5 shows sealed nasal interface 16 with two gas supply tubes (for redudancy and safety). In FIGS. 4-5, total system flow is represented by Qₛ; excess flow is represented by Qₑ (represented in FIG. 3 as Q_{e,inh} and Q_{e,exh} for excess flow during inhalation and exhalation, respectively); and nasopharyngeal pressure is represented by Pₙₐₛ. Other embodiments may include different designs for the nasal interface.

In some embodiments, the nasal interfaces 16 combine features from nasal pillows 27 and nasal prongs 29. In some embodiments, the nasal pillows (solid lined tubing in FIGS. 4-6) are configured to conform and seal the patient's nostrils and enable the recovery for excess gas, Qₑ, during the inhalation phase. In some embodiments, the nasal prongs (dotted/dashed lined tubing in FIGS. 4-6), which are integrated as secondary tubing elements within the nasal pillow mask, deliver the high-flow oxygen-rich gas, Qₛ.

FIG. 6 shows an embodiment of HFNT system 10 with oxygen recovery that uses the proposed sealed nasal interface 16 in FIGS. 4-5. In some embodiments, inspiration sensing algorithms 25 use readings from the nasopharyngeal pressure manometer/sensor for detecting the inspiratory phase of each breath cycle. In turn, the inspiration sensing algorithms 25 control valve 20 that directs the inspiratory gas into air/oxygen blender 11 for recovery (via O₂ sensor 23), while they disperse the expiratory gas into the ambient.

In some embodiments, system 10 includes oxygen supply 14 that is configured to deliver oxygen-enriched breathing gas to the patient during a breath cycle, wherein the breath cycle comprises an inspiratory phase and an expiratory phase; patient interface 16 that is configured to deliver the oxygen-enriched breathing gas to a nasal cavity of the patient; and valve 20 operatively connected patient interface 16 and oxygen supply 14, wherein valve 20 is configured to move between a first configuration and a second configuration based on an increase in pressure generated during the expiratory phase. When valve 20 is in the first configuration, valve 20 directs an excess flow of the oxygen-enriched breathing gas during the inspiratory phase to oxygen supply 14, and when valve 20 is in the second configuration, valve 20 vents an exhalation flow of patient 12 during the expiratory phase to atmosphere.

In some embodiments, the valve 20 may be a flap valve. For example, in one embodiment, the flap valve/gate opens when there is an excess flow during the exhalation phase and closes during the inhalation phase (or vice versa). That is, in one embodiment, the passive element (flap valve/other mechanical valves/membrane/gate) in the nasal cannula is configured to open to disperse the exhaled gas into the atmosphere and then close during the inhalation phase for recovery. In one embodiment, the flap valve may be referred to as a passive element that operates without a need of a computerized control system, while the electronic valve described above may be referred to as an active element. In one embodiment, the flap valve is a flow-dependent, one-way valve.

In some embodiments, control subsystem 116 is configured to control valve 20 to move between the first configuration and the second configuration based on the information as to whether patient 12 is in the inspiratory phase or patient 12 is in the expiratory phase. In some embodiments, control subsystem 116 is configured to control valve 20 to move between the first configuration and the second configuration based on output signals generated by one or more sensor 18 conveying information related to one or more parameters of the oxygen-enriched breathing gas being delivered to the nasal cavity of the patient. In some embodiments, control subsystem 116 is configured to provide input to valve 20 based on the determined one or more parameters of the oxygen-enriched breathing gas. In some embodiments, the provided input causing movement of valve 20 between the first configuration and the second configuration.

In some embodiments, one or more hardware/physical processors 24 are further configured to control valve 20 to move between the first configuration and the second configuration.

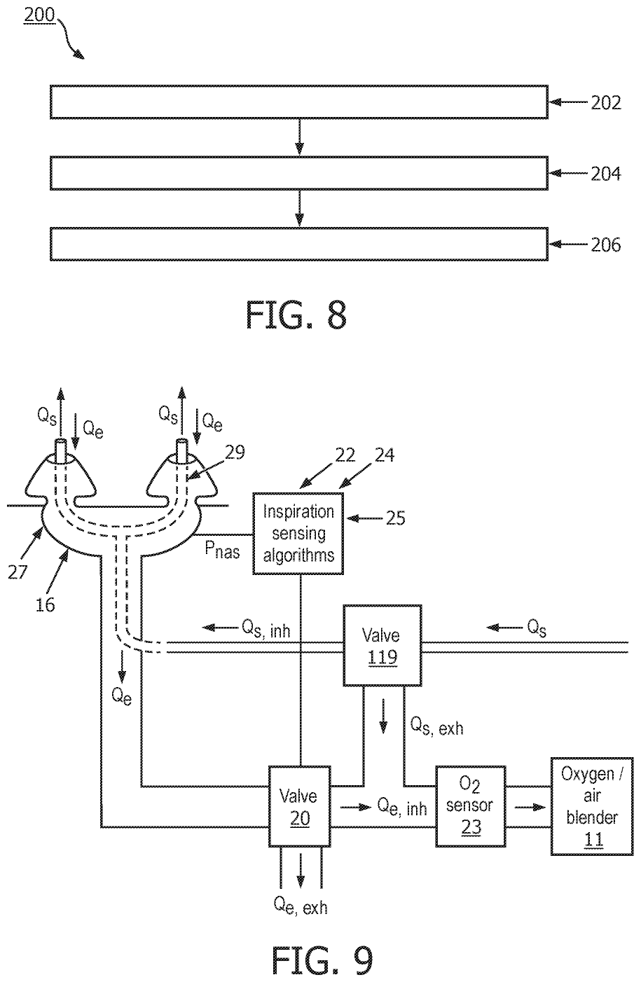

Referring to FIG. 8, a method 200 for providing oxygen therapy to patient 12 is provided. Method 200 is implemented by computer system 22 that comprises one or more physical/hardware processors 24 executing computer program/machine readable instructions that, when executed, perform method 200. In some embodiments, method 200 comprises providing, using oxygen supply 14 and patient interface 16, oxygen-enriched breathing gas to a nasal cavity of the patient during a breath cycle at procedure 202; obtaining, from one or more sensors 18, output signals conveying information related as to whether the patient is in the inspiratory phase or in the expiratory phase at procedure 204; and providing input to valve 20 based on the output signals, the provided input causing movement of the valve between a first configuration and a second configuration at procedure 206. The breath cycle comprises an inspiratory phase and an expiratory phase. When in the first configuration, the valve directs an excess flow of the oxygen-enriched breathing gas during the inspiratory phase to an oxygen supply. When in the second configuration, the valve vents an exhalation flow of the patient during the expiratory phase to atmosphere.

In some embodiments, method 200 further comprises obtaining, from one or more sensors 18, the output signals conveying information related to one or more parameters of the oxygen-enriched breathing gas being delivered to the nasal cavity of patient 12; determining the one or more parameters of the oxygen-enriched breathing gas based on the information in the output signals; and providing input to valve 20 based on the determined one or more parameters of the oxygen-enriched breathing gas, the provided input causing movement of valve 20 between the first configuration and the second configuration. In some embodiments, the one or more parameters of the oxygen-enriched breathing gas includes nasopharyngeal pressure.

In some embodiments, method 200 further comprises measuring, using oxygen sensor 23, oxygen fraction in the excess flow of the oxygen-enriched breathing gas, during the inspiratory phase, directed to oxygen supply 14.

In some embodiments, method 200 further comprises controlling, using air pump 15, flow rate of the oxygen-enriched breathing gas being delivered to the nasal cavity of patient 15; controlling, using oxygen blender 11, FiO₂ level of the oxygen-enriched breathing gas being delivered to the nasal cavity of patient 12; and heating and humidifying, using active humidifier 13, the oxygen-enriched breathing gas being delivered to the nasal cavity of patient 12.

In some embodiments, method 200 further comprises selecting by patient 12, using user interface 21 and according to a prescribed treatment plan, a predetermined flow rate flow rate of the oxygen-enriched breathing gas being delivered to the nasal cavity of patient 12; a predetermined FiO₂ level of the oxygen-enriched breathing gas being delivered to the nasal cavity of patient 12; and a predetermined temperature of the oxygen-enriched breathing gas being delivered to the nasal cavity of patient 12.

In some embodiments, the various computers and subsystems illustrated in FIGS. 2-3 and 6 may comprise one or more computing devices that are programmed to perform the functions described herein. The computing devices may include one or more electronic storages (e.g., database 132, or other electronic storages), one or more physical processors programmed with one or more computer program instructions, and/or other components. The computing devices may include communication lines or ports to enable the exchange of information with a network (e.g., network 150) or other computing platforms via wired or wireless techniques (e.g., Ethernet, fiber optics, coaxial cable, WiFi, Bluetooth, near field communication, or other communication technologies). The computing devices may include a plurality of hardware, software, and/or firmware components operating together to provide the functionality attributed herein to the servers. For example, the computing devices may be implemented by a cloud of computing platforms operating together as the computing devices.

The electronic storages may comprise non-transitory storage media that electronically stores information. The electronic storage media of the electronic storages may include one or both of system storage that is provided integrally (e.g., substantially non-removable) with the servers or removable storage that is removably connectable to the servers via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storages may store software algorithms, information determined by the processors, information received from the servers, information received from client computing platforms, or other information that enables the servers to function as described herein.

The processors may be programmed to provide information processing capabilities in the servers. As such, the processors may include one or more of a digital processor, an analog processor, or a digital circuit designed to process information, an analog circuit designed to process information, a state machine, and/or other mechanisms for electronically processing information. In some embodiments, the processors may include a plurality of processing units. These processing units may be physically located within the same device, or the processors may represent processing functionality of a plurality of devices operating in coordination. The processors may be programmed to execute computer program instructions to perform functions described herein of subsystems 112, 114, 116 or other subsystems. The processors may be programmed to execute computer program instructions by software; hardware; firmware; some combination of software, hardware, or firmware; and/or other mechanisms for configuring processing capabilities on the processors.

It should be appreciated that the description of the functionality provided by the different subsystems 112-116 described herein is for illustrative purposes, and is not intended to be limiting, as any of subsystems 112-116 may provide more or less functionality than is described. For example, one or more of subsystems 112-116 may be eliminated, and some or all of its functionality may be provided by other ones of subsystems 112-116. As another example, additional subsystems may be programmed to perform some or all of the functionality attributed herein to one of subsystems 112-116.

It should be appreciated that the different subsystems 112-116 performing the operations illustrated in FIG. 2 may reside in system 10 itself. In other embodiments, the different subsystems 112-116 performing the operations illustrated in FIG. 2 may reside in an independent monitoring device.

In some embodiments, system 10 may be used in home healthcare solutions or systems. In some embodiments, system 10 may be used in home respiratory/oxygen systems. In some embodiments, system 10 may be used for mild to moderate chronic obstructive pulmonary disease (COPD) patients. In some embodiments, system 10 may be used for obstructive sleep apnea (OSA) patients. In some embodiments, the continuous flow of air during HFNT delivered to patient 12 provides some extra benefits, like CO₂ clearance, small PEEP, etc.

In some embodiments, system 10 may also include a communication interface that is configured to send the determined control signals to adjust valve 20 through an appropriate wireless communication method (e.g., Wi-Fi, Bluetooth, internet, etc.) or send to other systems for further processing. In some embodiments, system 100 may include a recursive tuning subsystem that is configured to recursively tune its intelligent decision making subsystem using available data or information to provide better overall adjustment of valve 20. In some embodiments, intelligent decision making subsystem, communication interface and recursive tuning subsystem may be part of computer system 22 (comprising server 24).

The current/prior art HFNT systems disperse excess gas to ambient. On the other hand, the present patent application describes a novel HFNT system where oxygen-rich gas is being recovered and utilized for improving the overall system's efficiency. The systems and methods of the present patent application are used in home ventilation business and/or critical care ventilation business.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system configured to provide oxygen therapy to a patient, the system comprising:
an oxygen supply configured to provide oxygen-enriched breathing gas to the patient during a breath cycle, the breath cycle comprising an inspiratory phase and an expiratory phase;
a patient interface configured to deliver the oxygen-enriched breathing gas to a nasal cavity of the patient;
one or more sensors configured to generate output signals conveying information as to whether the patient is in the inspiratory phase or in the expiratory phase;
one or more valves operatively associated with the patient interface and the oxygen supply, the one or more valves having a) a first configuration in which the one or more valves operate to recover an excess flow of the oxygen-enriched breathing gas during the inspiratory phase, and b) a second configuration in which the one or more valves vent an exhalation flow of the patient during the expiratory phase to atmosphere; and
a computer system that comprises one or more physical processors operatively connected with the one or more sensors and the one or more valves, the one or more physical processors being programmed with computer program instructions which, when executed cause the computer system to provide input to the one or more valves based on the output signals, the provided input causing movement of the one or more valves between the first configuration and the second configuration.

2. The system of claim 1, wherein the one or more sensors are configured to generate the output signals conveying information related to one or more parameters of the oxygen-enriched breathing gas being delivered to the nasal cavity of the patient, and
wherein the one or more physical processors are configured to determine the one or more parameters of the oxygen-enriched breathing gas based on the information in the output signals; and provide input to the one or more valves based on the determined one or more parameters of the oxygen-enriched breathing gas, the provided input causing movement of the one or more valves between the first configuration and the second configuration.

3. The system of claim 2, wherein the one or more parameters of the oxygen-enriched breathing gas includes nasopharyngeal pressure.

4. The system of claim 1, wherein the patient interface includes a seal structure constructed and arranged to form a seal with a region surrounding an entrance to the nasal cavity of the patient such that the oxygen-enriched breathing gas is delivered to the nasal cavity of the patient and the excess flow of the oxygen-enriched breathing gas, during the inspiratory phase, is directed to the oxygen supply.

5. The system of claim 1, wherein the system further comprises an oxygen sensor configured to measure oxygen fraction in the excess flow of the oxygen-enriched breathing gas, during the inspiratory phase, directed to the oxygen supply.

6. The system of claim 1, wherein, when the one or more valves are in the first configuration, the one or more valves are configured to direct the excess flow of the oxygen-enriched breathing gas during the inspiratory phase to the oxygen supply.

7. The system of claim 1, wherein, when the one or more valves are in the first configuration, the one or more valves are configured to recirculate the excess flow of the oxygen-enriched breathing gas during the inspiratory phase to the patient

8. A system configured to provide oxygen therapy to a patient, the system comprising:
an oxygen supply configured to deliver oxygen-enriched breathing gas to the patient during a breath cycle, the breath cycle comprising an inspiratory phase and an expiratory phase;
a patient interface configured to deliver the oxygen-enriched breathing gas to a nasal cavity of the patient; and
one or more valves operatively connected the patient interface and the oxygen supply, the one or more valves configured to move between a first configuration and a second configuration based on an increase in pressure generated during the expiratory phase,
wherein, when the one or more valves are in the first configuration, the one or more valves operate to recover an excess flow of the oxygen-enriched breathing gas during the inspiratory phase, and
wherein, when the one or more valves are in the second configuration, the one or more valves vent an exhalation flow of the patient during the expiratory phase to atmosphere.

9. A method for providing oxygen therapy to a patient, the method being implemented by a computer system that comprises one or more physical processors executing machine readable instructions that, when executed, perform the method, the method comprising:
providing, using an oxygen supply and a patient interface, oxygen-enriched breathing gas to a nasal cavity of the patient during a breath cycle, the breath cycle comprising an inspiratory phase and an expiratory phase, the patient interface being configured to recover the excess gases during the inhalation phase and the exhalation phase;
obtaining, from one or more sensors, output signals conveying information related as to whether the patient is in the inspiratory phase or in the expiratory phase; and
providing input to one or more valves based on the output signals, the provided input causing movement of the one or more valves between a first configuration and a second configuration,
wherein, when in the first configuration, the one or more valves operate to recover an excess flow of the oxygen-enriched breathing gas during the inspiratory phase, and
wherein, when in the second configuration, the one or more valves vent an exhalation flow of the patient during the expiratory phase to atmosphere.

10. The method of claim 9, wherein obtaining, from the one or more sensors, the output signals conveying information related to one or more parameters of the oxygen-enriched breathing gas being delivered to the nasal cavity of the patient;
determining the one or more parameters of the oxygen-enriched breathing gas based on the information in the output signals; and
providing input to the one or more valves based on the determined one or more parameters of the oxygen-enriched breathing gas, the provided input causing movement of the one or more valves between the first configuration and the second configuration.

11. The method of claim 10, wherein the one or more parameters of the oxygen-enriched breathing gas includes nasopharyngeal pressure.

12. The method of claim 9, wherein the patient interface includes a seal structure constructed and arranged to form a seal with a region surrounding an entrance to the nasal cavity of the patient such that the oxygen-enriched breathing gas is delivered to the nasal cavity of the patient and the excess flow of the oxygen-enriched breathing gas, during the inspiratory phase, is directed to the oxygen supply.

13. The method of claim 9, further comprising measuring, using an oxygen sensor, oxygen fraction in the excess flow of the oxygen-enriched breathing gas, during the inspiratory phase, directed to the oxygen supply.

14. The method of claim 9, wherein, when the one or more valves are in the first configuration, the one or more valves are configured to direct the excess flow of the oxygen-enriched breathing gas during the inspiratory phase to the oxygen supply.

15. The method of claim 9, wherein, when the one or more valves are in the first configuration, the one or more valves are configured to recirculate the excess flow of the oxygen-enriched breathing gas during the inspiratory phase to the patient.
